(19) United States
(12) Patent Application Publication
(10) Pub. No.: US 2026/0266743 A1
(43) Pub. Date: Sep. 10, 2026

(54) APPARATUS AND METHOD FOR INSPECTING CONTAINER BY X-RAY RADIATION

(71) Applicant: KRONES AG, Neutraubling (DE)

(72) Inventor: Thorsten GUT, Neutraubling (DE)

(21) Appl. No.: 18/873,279

(22) PCT Filed: Mar. 30, 2023
(86) PCT No.: PCT/EP2023/058379

§ 371 Date: Dec. 9, 2024

(30) Foreign Application Priority Data
Jun. 8, 2022 (DE) 10 2022 114 371.6
Jun. 8, 2022 (DE) 20 2022 103 221.1

(51) Int. Cl. G01N 23/04; G01N 33/00; G01N 35/04

(52) U.S. Cl. CPC G01N 23/04; G01N 33/0081; G01N 35/04; G01N 2035/0465; G01N 2035/0484; G01N 2223/1016; G01N 2223/309; G01N 2223/501; G01N 2223/623; G01N 2223/639; G01N 2223/643

(57) ABSTRACT

Apparatus for inspecting containers, with a transport device which transports the containers along a predefined transport direction, with at least a first radiation device which emits high-energy radiation and in particular X-ray radiation onto the containers that are to be inspected, and with a first sensor device which is configured for receiving the radiation emitted onto the containers by the radiation device, wherein the transport device forms a support surface which is movable in the transport direction and which supports the containers at their container bottoms, characterized in that the support surface has a large number of carrier elements made of a plastic material and moreover has at least one guide device for guiding these carrier elements, wherein this guide device is formed of a metal or a metal-reinforced plastic.

The present invention relates to an apparatus and a method for inspecting containers, and in particular filled containers. A variety of apparatuses and methods for inspecting containers are known from the prior art. Apparatuses and methods are also known in which in particular the bottom regions or the bottoms of such containers and in particular also filled containers are inspected. For this purpose, it is known to carry out such inspections using X-ray radiation. The biggest problem with this full bottle inspection using X-ray radiation is the transport through the X-ray unit. It is known that containers are transported with two side belts and are therefore exposed on the bottom. It is also known that the containers are transported on a transport chain or a conveyor belt.

In order for the measurement of the container bottom to work, the transport chain should be very homogeneous and preferably free of metal. For example, a special chain with solid plastic elements is known from the state of the art. The problem with these chains is that they are prone to wear and tear; for example, the chain stretches or wears out quite quickly and therefore has to be replaced often.

In addition, despite the special construction, remnants of structures are still visible in the camera image because the plastic used absorbs a significant part of the X-ray radiation and the chain becomes visible as a “ghost image,” so to speak. The use of a less absorbed plastic cannot be used in the prior art because it is too soft to function as a chain and stretches and has to be replaced even faster. This also leads to a short service life.

From EP 2435344B1 , an X-ray inspection system with a conveyor chain is known.

The present invention is therefore based upon the object of providing an apparatus and a method for inspecting containers which, on the one hand, exhibit little wear and, on the other hand, allow proper inspection by X-ray radiation. This is achieved according to the invention by the subject matter of the independent claims. Advantageous embodiments and developments are the subject matter of the dependent claims.

An apparatus according to the invention for inspecting containers has a transport device which transports the containers along a predefined transport direction. In addition, the apparatus has at least one first radiation device which emits high-energy radiation and in particular X-ray radiation onto the containers to be inspected, and a first sensor device which is suitable and intended for receiving the radiation emitted onto the containers by the radiation device. The transport device has a support surface (and/or support device) which is movable in the transport direction and which supports the containers at their container bottoms and in particular transports them in this way.

According to the invention, the support surface has a plurality of carrier elements made of a plastic material and moreover has at least one guide device for guiding these carrier elements, wherein this guide device has a metal. The guide device can be made of a metal or can have a metal for example, it can be made of a metal-reinforced plastic. The metal is preferably selected from a group of metals which includes iron, steel, and aluminum, but other metals may also be considered.

The idea of the invention is therefore to create a carrier element for the containers which is built in two parts. Those regions which serve to directly support the container are preferably formed from a non-absorbent plastic, and the elements which are subject to high loads, e.g., the guide elements mentioned, are produced from a harder material, wherein it is accepted that this material also absorbs X-ray radiation.

Preferably, the support surface is flexible, which is achieved in particular by a mobility of the individual carrier elements with respect to one another. Preferably, this flexibility exists in a plane which is formed by the transport direction and a longitudinal direction of the transported containers.

Preferably, the transport device transports the containers in an upright position, wherein the bottoms of the containers stand in particular on the support surface.

On the one hand, this ensures that the X-ray radiation is not absorbed in the region relevant for the inspection, and thus the inspection is not impaired, but on the other hand, those elements of the transport device which are subject to high mechanical loads are made of a more stable material.

A plastic with very low X-ray absorption is therefore used as the material for the carrier elements for the containers. These carrier elements or plates are preferably guided by a guide device, such as a wide transport station, which can have toothed belts and in particular steel and/or CFRP-reinforced toothed belts as guide elements, as described in more detail below.

Preferably, the carrier elements are each fastened to the guide device (which can also be referred to as a carrier device).

Particularly preferably, the apparatus has a shielding device for shielding against X-ray radiation. Particularly preferably, this shielding device completely surrounds the apparatus in the transport direction of the containers, in particular in the inspection region. This shielding device can have walls that prevent X-ray radiation from passing through. In addition, this shielding device can also have a labyrinth-like structure which prevents the X-ray radiation from escaping from the shielding device after one or preferably two reflections.

The carrier elements in question do not absorb the X-ray radiation or absorb it only to a small extent. Although the guide device absorbs X-ray radiation, but it is preferably arranged outside the measuring field for the plastic containers and therefore does not influence the inspection.

Preferably, the carrier elements are arranged on the upper side of the support surface carrying the container bottoms, and the guide devices are arranged on the underside of the carrier elements opposite this upper side.

The transport direction is particularly preferably straight. Preferably, the radiation and in particular X-ray radiation is directed at least also onto the container bottoms and in particular passes through them. The receiver device, or sensor device, detects radiation that has passed through the containers.

In a preferred embodiment, the sensor device is suitable and intended for spatially resolved recording of the X-ray radiation. Particularly preferably, the sensor device is suitable for recording and/or outputting a spatially resolved image of the (X-ray) radiation incident upon it.

Particularly preferably, the guide device or components of the guide device are not located in the beam path of the outgoing X-ray radiation to be received.

In a further advantageous embodiment, the carrier elements have a parallelogram-shaped cross-section with respect to a plane which extends perpendicular to the transport surface and in the transport direction. This preferably ensures that adjoining sides of adjacent carrier elements are inclined at an oblique angle with respect to the transport direction, and preferably the carrier elements overlap each other in the transport direction.

In a further preferred embodiment, the guide devices are arranged outside the transport region of the transport device. This means that the containers to be transported are not arranged above the guide devices.

In a further preferred embodiment, the transport device is operable such that containers to be inspected are transported through a curtain of electromagnetic radiation, wherein the curtain of electromagnetic radiation extends in a plane which crosses the plane of the support surface along a line which runs perpendicular to the transport direction.

Particularly preferably, the transport device has a transport length (in particular in the transport direction) that is greater than 1 m, preferably greater than 2 m, preferably greater than 3 m, and preferably greater than 3.5 m. In a further advantageous embodiment, the transport device has a transport length in the transport direction that is less than 10 m, preferably less than 8 m, preferably less than 6 m, and preferably less than 5 m.

In a further preferred embodiment, the carrier elements are constructed in the form of webs or strips and preferably have a rectangular or parallelogram-like cross-section.

In a further preferred embodiment, the carrier elements have a first end portion and a second end portion, and the guide devices are arranged on these end portions and in particular fastened thereto. In this way, on the one hand, a high level of stability can be achieved, and on the other hand, it can be prevented that the guide devices be located within a measuring region of the apparatus.

In a further advantageous embodiment, the carrier elements are made of a plastic which does not absorb X-ray radiation or absorbs it only to a very small extent. Particularly preferably, the plastic material is selected from a group of plastic materials which includes PEEK, Teflon, and PET. In a further preferred embodiment, the carrier elements are produced from a synthetic material that is permeable to high-energy radiation, in particular of acetate resin or polypropylene.

Particularly preferably, two-sided guide and/or holding devices are provided for the carrier elements. It is possible that the carrier elements be fixed to these guide devices. Particularly preferably, the (in particular, lateral) guide devices are arranged at a distance from one another which is at least 10 cm, preferably at least 20 cm, and preferably at least 25 cm. Preferably, these guide devices are at a distance from each other that is less than 60 cm, preferably less than 50 cm, preferably less than 40 cm. These distances have proven to be particularly favorable for ensuring sufficient stability, on the one hand, and keeping the guide devices out of the measuring field of the apparatus, on the other hand.

In a preferred embodiment, the at least one radiation device is arranged laterally of the transport station and/or transport device.

In a further advantageous embodiment, the transport device is a circulating transport device and in particular a circulating conveyor belt or a transport chain. This means that the individual chain links are preferably composed of the guide devices and the carrier elements arranged on them. The guides of this chain are therefore, as mentioned above, arranged laterally or partially laterally with respect to the carrier elements.

Preferably, a distance between the upper side of the conveyor belt running in the transport direction (which at this moment also forms the support surface) and the underside of the conveyor belt running counter to the transport direction is greater than 10 cm, preferably greater than 20 cm, preferably greater than 30 cm. Particularly preferably, this distance between the upper side of the conveyor belt running in the transport direction and the underside running counter to the transport direction is less than 100 cm, preferably less than 90 cm, preferably less than 80 cm, and preferably less than 70 cm.

In a further advantageous embodiment, at least one radiation device is arranged between the upper side and the underside of the transport devices.

In a further advantageous embodiment, the guide device is a belt and in particular a toothed belt, or has a belt and in particular a toothed belt. In particular, this is a steel-reinforced toothed belt. As mentioned above, two guide devices, in particular two lateral toothed belts, are particularly preferably provided. The carrier elements can be fastened to these.

In a further advantageous embodiment, the carrier elements have a thickness that is greater than 2 mm and preferably greater than 3 mm. In a further advantageous embodiment, the carrier elements have a thickness that is less than ten mm, preferably less than 8 mm, and particularly preferably less than 6 mm. Particularly preferably, this thickness is substantially constant over the width of the carrier elements.

In a further advantageous embodiment, the carrier elements have a length, running perpendicular to the transport direction, that is greater than 10 cm, preferably greater than 20 cm, preferably greater than 25 cm. In a further preferred embodiment, the carrier elements have a length, running perpendicular to the transport direction, that is less than 100 cm, preferably 90 cm, preferably 80 cm, preferably 70 cm, preferably 60 cm, preferably 50 cm, preferably 40 cm, and particularly preferably less than 30 cm.

As mentioned above, these lengths are well chosen because, on the one hand, they still have sufficient stability, and, on the other hand, they are wide enough to avoid disruptive influences.

Particularly preferably, at the position where the containers are located on them, the carrier elements are at a distance from each other that is less than 10 mm, preferably less than 8 mm, preferably less than 6 mm, preferably less than 4 mm, and preferably less than 3 mm.

In a further advantageous embodiment, the carrier elements have a width in the transport direction that is greater than 5 cm, preferably greater than 10 cm, and preferably greater than 15 cm. Particularly preferably, the carrier elements have a width in the transport direction that is less than 50 cm, preferably less than 40 cm, preferably less than 30 cm, and preferably less than 25 cm. These dimensions have also proven to be particularly advantageous for being able to operate the apparatus without problems.

In a preferred embodiment, the apparatus has at least one second (X-ray) radiation device and/or at least one second receiving device. Particularly preferably, at least one second (X-ray) radiation device and one second receiving and/or sensor device are provided. These can inspect the container to be inspected preferably from different angles.

Particularly preferably, at least one X-ray radiation device is arranged above the bottom of the containers, and particularly preferably at least one X-ray radiation device is arranged below the bottom of the containers. Particularly preferably, at least one sensor device is also arranged above the bottom of the containers, and/or at least one sensor device is arranged below the bottom of the containers. In this way, a comprehensive image of the bottom of each container can be recorded using the transmitted light method.

Particularly preferably, at least one X-ray radiation device and a sensor device, which receives the radiation radiated onto the containers by this X-ray radiation device, are arranged such that a beam path of the X-ray radiation runs obliquely with respect to a longitudinal direction of the containers to be inspected. Particularly preferably, this beam path also runs obliquely with respect to the transport direction of the containers. Particularly preferably, an angle between the longitudinal direction of the containers and the main beam direction of the X-ray radiation device is more than 10°, preferably more than 15°, preferably more than 20°, preferably more than 25°, and preferably more than 30° and preferably more than 35°. Particularly preferably, this angle is less than 80°, preferably less than 70°, preferably less than 65°, preferably less than 60°, and particularly preferably less than 55°.

In a further preferred embodiment, at least one X-ray radiation device and the sensor device, which receives the radiation radiated onto the containers by the X-ray radiation device, are arranged such that a beam path of the X-ray radiation runs obliquely with respect to a longitudinal direction of the containers to be inspected. In a preferred embodiment, the radiation device or devices are arranged laterally with respect to the transport device and can thus take the recordings through this plastic-air (and in particular PEEK-air) region. Preferred recording angles are, for example, 45° angles obliquely from above. The only dead zone here is the narrow belt, which is arranged far outside the container bottom.

In a preferred embodiment, the guide devices and/or sliding profiles within the measuring window are also made of plastic, and in particular PEEK. Before and after this measuring window, standard elements (e.g., made of steel and/or reinforced plastic) are again provided.

In a further advantageous embodiment, the apparatus has a discharge device which discharges the inspected containers from the transport device. A transfer device which transfers the containers from the transport device to the discharge device is preferably provided here.

Particularly preferably, this transfer device is configured in such a way that it transports the containers obliquely to the transport direction. Particularly preferably, a transport angle relative to the transport direction of more than 5°, preferably more than 10°, preferably more than 15°, and preferably more than 20° is provided. Preferably, this angle is less than 60°, preferably less than 50°, preferably less than 40°.

Particularly preferably, the transfer device is a railing or has a railing, which particularly preferably transfers laterally down onto the discharge device, in particular a further transport device, such as a normal slatband chain.

Particularly preferably, a position detection device and/or a path tracking is provided downstream of the radiation devices in the transport direction. Particularly preferably, this position detection device has at least one light barrier and preferably a light barrier array, in particular for FIFO tracking (first in/first out). Particularly preferably, the apparatus has at least one further position detection device and preferably several further position detection devices. As mentioned, these can, for example, be light barriers.

In a further advantageous embodiment, the apparatus has a feed device which conveys the containers to the transport device, wherein this feed device preferably has a transport wheel conveying the containers or a transport screw conveying the containers.

It is also possible that the insertion, as mentioned above, take place as a displacement from a normal conveyor belt. In this described embodiment, however, the containers are placed directly onto the transport device and in particular positioned centrally by a dividing system, such as a transport wheel. This dividing system can, for example, be a dividing screw which is arranged obliquely, or an outlet starwheel of a preceding rotary conveyor such as a filling device or a labeling machine, or even a one-sided or two-sided belt station.

The present invention is further directed towards a plant for handling containers for liquid. It preferably has a filling device and in particular also a labeling or printing device for printing the containers. An apparatus according to the invention, as described above, for inspecting the containers is arranged downstream of these devices. In a further advantageous embodiment, the present invention is used in a plant for filling cans.

The present invention is further directed to a method for inspecting containers, wherein the containers are transported along a predefined transport direction with a transport device, and at least one first X-ray radiation device emits X-ray radiation onto the containers to be inspected, and a first sensor device detects radiation emitted onto the containers by the X-ray radiation device or radiation resulting therefrom (or radiation radiated onto the containers by the radiation device and transmitted by the containers), wherein the transport device forms a support surface which is movable in the transport device and which supports the containers at their container bottoms and thus transports them.

According to the invention, the support surface has a plurality of carrier elements made of a plastic material (and in particular a plastic material that does not absorb X-ray radiation or only slightly absorbs it) and moreover at least one guide device for guiding these carrier elements, wherein this guide device is reinforced and/or has a metal and/or an X-ray radiation-absorbing material.

Preferably, the containers are filled and, in particular, also labeled and/or printed containers. In particular, the containers are containers closed with container closures or filled, closed beverage cans.

Preferably, the containers are transported by the transport device in such a way that they are at a distance from one another in the transport direction. Preferably, this distance between the containers is greater than 2 mm, preferably greater than 3 cm, preferably greater than 4 cm, and preferably greater than 5 cm. Preferably, this distance is less than 30 cm, preferably less than 20 cm, preferably less than 15 cm.

In a further preferred method, the containers are transported in a straight line during their inspection and/or are transported at least also perpendicular to the transport direction after their inspection.

In a further preferred method, the containers are fed to the transport device with a conveyor belt or a rotary conveyor, a dividing screw, or a one-sided or two-sided belt station. Particularly preferably, the containers are discharged after their inspection with a further conveyor belt.

In a further preferred method, the sensor device records a spatially resolved image of the transported containers.

Particularly preferably, the bottoms of the containers are inspected. Particularly preferably, the sensor device receives radiation emitted by the X-ray radiation device and transmitted by the containers.

Particularly preferably, the X-ray radiation therefore passes through the carrier elements between the radiation device and the sensor device.

Preferably, in the context of the invention, it is proposed to use a plastic, and in particular Lexan or PET, as the carrier element.

In the context of the invention, it is therefore particularly preferred proposed, to use a plastic, and in particular PEEK, as the carrier element. PEEK is almost invisible in a camera image and has almost the same absorption as air at the X-ray-specific wavelength. In this way, it is also possible for the carrier elements to not be extremely close together and formed with no air gap.

A further advantage results from the above-described, in particular lateral, loading and/or discharging of the containers, in particular in the inlet and/or outlet region. In this way, X-ray protection is simultaneously achieved in the transport direction of the apparatus.

If, as described above, an outlet starwheel of a rotary conveyor is used in the inlet region, it is possible to make the entire inspection apparatus very compact and, in particular, to mount it on or integrate it into a guiding machine. Preferably, the service life of the transport device is also significantly longer than that of a conventional transport chain.

Further advantages and embodiments can be seen in the accompanying drawings, in which:

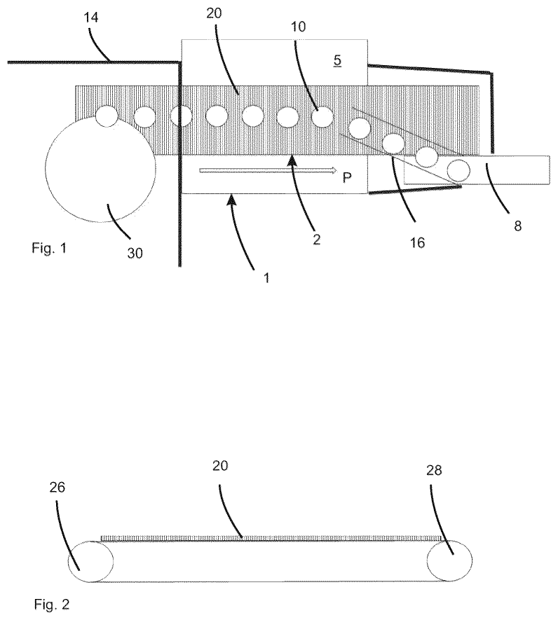

FIG. 1 is a schematic representation of an apparatus according to the invention;

FIG. 2 is a side view of the apparatus shown in FIG. 1;

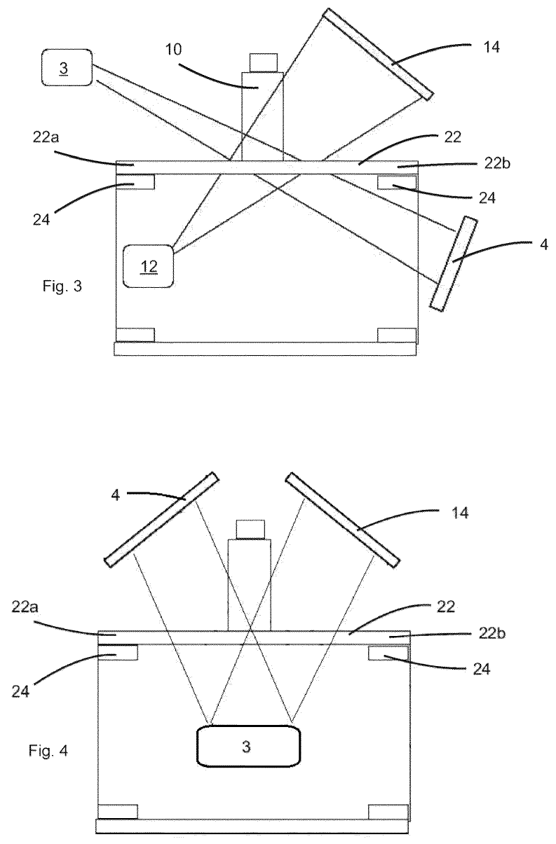

FIG. 3 is a detail view of the inspection using X-ray radiation; and

FIG. 4 is a further detail view of the inspection using X-ray radiation.

FIG. 1 shows a schematic representation of an apparatus 1 according to the invention for inspecting containers 10. Reference sign 2 designates a transport device, such as a conveyor belt or a transport chain, which transports the containers 10 in the transport direction P. Reference sign 14 designates a shield, which serves to shield against X-ray radiation. Reference sign 5 schematically designates the actual X-ray inspection device, which is described in more detail below. After their inspection, the containers 10 are conveyed by a transfer device 16 to a further transport device, or a discharge device 8, which can again be a conveyor belt. It can be seen that the transfer device 16 transports the containers not only in the transport direction P, but also perpendicular to it.

Reference sign 30 designates a feed device, such as a transport starwheel, which deposits the containers 10—, substantially centrally onto the transport device 2. Reference sign 20 designates the support surface which supports the containers during their transport.

FIG. 2 shows a partial side view of the apparatus shown in FIG. 1. In this case, deflection rollers 26, 28 are provided which guide the transport means (i.e., in particular the support surface and the guide devices) in a circumferential manner. Here again, the support surface 20 is shown, but only on the upper side. This support surface 20 is composed of a plurality of carrier elements (described in more detail below).

FIG. 3 shows a further view of the actual inspection unit 5. Reference sign 3 refers here to a first X-ray radiation device, and reference sign 12 refers to a second X-ray radiation device. These each illuminate the support surface and in particular the individual carrier elements 22. Reference signs 4 and 14 designate sensor devices, which detect the radiation directed onto the containers by the radiation device and, in particular, detect it in a spatially resolved manner. These sensor devices can have cameras that are sensitive to X-ray radiation.

It can be seen that the carrier element 22 has two lateral guide devices 24. Reference signs 22A and 22B designate the end portions of the carrier elements, which are carried by the guide device 24. The guide devices 24 are made of a material which also absorbs X-ray radiation. However, as shown in FIG. 3, the regions of the container bottom and also of the carrier element 22 detected by the sensor devices are outside the regions in which the guide devices 24 are located.

FIG. 4 shows a further representation of an inspection unit in a further embodiment. In this embodiment, only one X-ray radiation device 3 is provided, which, however, preferably radiates in several directions. This X-ray radiation device 3 is preferably arranged here below the containers.

Furthermore, two sensor devices 4 and 14 are provided here, which are preferably both arranged above the transport device and/or above the bottoms of the containers. The transport device is configured here in the same way as in the embodiment shown in FIG. 3.

The applicant reserves the right to claim all features disclosed in the application documents as essential to the invention, provided that they are novel over the prior art individually or in combination. It is also pointed out that features which can be advantageous in themselves are also described in the individual figures. A person skilled in the art will immediately recognize that a particular feature described in a figure can be advantageous even without the adoption of further features from this figure. Furthermore, the person skilled in the art will recognize that advantages can also result from a combination of several features shown in individual or in different figures.

1: An apparatus for inspecting containers, with a transport device which is configured to transport the containers along a predefined transport direction, with at least one first radiation device which is configured to emit high-energy radiation and in particular X-ray radiation onto the containers to be inspected, and with a first sensor device which is configured for receiving the radiation emitted onto the containers by the radiation device, wherein the transport device forms a support surface which is configured to be movable in the transport direction and to support the containers at their container bottoms,
wherein
the support surface has a plurality of carrier elements made of a plastic material and moreover at least one guide device which is configured for guiding these carrier elements, wherein this guide device has a metal.

2: The apparatus according to claim 1,
wherein
the carrier elements are constructed in the form of webs or strips and preferably have a rectangular or parallelogram-like cross-section.

3: The apparatus according to claim 1,
wherein
the carrier elements have a first end portion and a second end portion, and the guide elements are arranged at these end portions.

4: The apparatus according to claim 1,
wherein
the carrier elements are made of a plastic material which does not absorb X-ray radiation or absorbs it only to a small extent, wherein the plastic material is preferably selected from a group of plastic materials which includes PEEK, Teflon, and PET.

5: The apparatus according to claim 1,
wherein
the transport device is a circulating transport device and in particular a conveyor belt or a transport chain.

6: The apparatus according to claim 1, wherein
wherein
the guide device is a belt and in particular a toothed belt.

7: The apparatus according to claim 1, wherein
wherein
the carrier elements have a thickness that is greater than 2 mm and preferably than 3 mm, and/or the carrier elements have a thickness that is less than 10 mm, preferably less than 8 mm, and particularly preferably less than 6 mm.

8: The apparatus according to claim 1,
wherein
the apparatus has at least one second radiation device and/or at least one second sensor device.

9: The apparatus according to claim 1,
wherein
at least one X-ray radiation device and the sensor device, which receives the radiation radiated onto the containers by the X-ray radiation device, are arranged such that a beam path of the X-ray radiation runs obliquely with respect to a longitudinal direction of the containers to be inspected.

10: The apparatus according to claim 1,
wherein
the apparatus has a discharge device which is configured to discharge the inspected containers from the transport device, wherein preferably a transfer device is provided which which is configured to transfer the containers from the transport device to the discharge device.

11: The apparatus according to claim 10,
wherein
this transfer device is configured to transport the containers obliquely to the transport direction.

12: The apparatus according to claim 1,
wherein
the apparatus has a feed device which is configured to convey the containers to the transport device, wherein this feed device preferably has a transport wheel which is configured to convey the containers or a transport wheel conveying the containers or a transport screw conveying the containers.

13: A method for inspecting containers, wherein the containers are transported along a predefined transport direction using a transport device, and at least one first radiation device which emits high-energy radiation and in particular X-ray radiation onto the containers to be inspected, and a first sensor device detects radiation emitted onto the containers by the radiation device, wherein the transport device forms a support surface which is movable in the transport direction and which supports the containers at their container bases,
wherein
the support surface has a plurality of carrier elements made of a plastic material and moreover at least one guide device for guiding these carrier elements, wherein this guide device has a metal.

14: The method according to claim 13,
wherein
the containers are filled and preferably also labeled and/or printed containers.

15: The method according to claim 13,
wherein
the containers are transported in a straight line during their inspection and/or are transported at least perpendicular to the transport direction after their inspection.

16: The apparatus according to claim 1, wherein the guide device is formed at least in part of a material which absorbs X-ray radiation.

17: The apparatus according to claim 16, wherein the material which absorbs X-ray radiation comprises a metal or a metal-reinforced plastic selected from the group consisting of iron, steel and aluminum.

18: The method according to claim 13, wherein the guide device is formed at least in part of a material which absorbs X-ray radiation.

19: The method according to claim 18, wherein the material which absorbs X-ray radiation comprises a metal or a metal-reinforced plastic selected from the group consisting of iron, steel and aluminum.
